(19) United States
(12) Patent Application Publication
(10) Pub. No.: US 2013/0312134 A1
(43) Pub. Date: Nov. 21, 2013

(54) WHITE HONEYDEW MELON

(71) Applicant: Jean Poulos, Lodi, CA (US)

(72) Inventor: Jean Poulos, Lodi, CA (US)

(21) Appl. No.: 13/615,721

(22) Filed: Sep. 14, 2012

Related U.S. Application Data
(60) Provisional application No. 61/647,699, filed on May 16, 2012.

(51) Int. Cl. A01H 5/08; A01G 1/00

(52) U.S. Cl. CPC A01H 5/08; A01G 1/001
USPC 800/260; 800/309; 800/303; 435/410; 47/58.1 FV; 426/615; 426/635

(57) ABSTRACT

The invention relates to the field of Cucumis melo, in particular to honeydew melon plants and honeydew melon fruits. A honeydew melon plant producing fruits having white fruit flesh is provided.

### FIELD OF THE INVENTION

The present invention relates to the field of plant breeding. In particular, a new and distinct hybrid variety of melon is provided, designated NUN 01052 MEM (also referred to as hybrid variety ‘Dolce Crema’ or ‘Dolce Crema F1’).

The new variety is a honeydew type (Cucumis melo) with white flesh and unique fruit aroma, flavor, texture and/or fruit storage properties.

Provided are seeds of NUN 01052 MEM, plants and plant parts produced from these seeds such as harvested fruit or parts thereof, pollen, cells, leaves or parts thereof, petioles, shoots or parts thereof, stems or parts thereof, roots or parts thereof, cuttings or parts thereof, flowers, vegetative reproductions of the variety NUN 01052 MEM, and progeny of the variety.

Melons or ‘muskmelons’, Cucumis melo, can be classified into: C. melo cantalupensis, C. melo inodorous and C. melo reticulatus.

C. melo cantalupensis are also referred to as Canteloupes and are primarily round in shape with prominent ribs and almost no netting. Most have orange, sweet flesh and they are usually very fragrant. In contrast to the European cantaloupe, the North American ‘Cantaloupe’ is not of this type, but belongs to the true muskmelons.

C. melo inodorous (or winter melons) can be subdivided into different types, such as Honeydew melon, Piel de Sapo, Sugar melon, Japanese melon, etc.

C. melo reticulatus is the true muskmelon, with reticulated skin (netted) and includes Galia melons, Sharlyn melons and the North American cantaloupe.

Melons come in many sizes and shapes including round, oval, and cylindrical. The flesh is generally orange and quite sweet, but some varieties of melon and specifically, the Persian melons, can have green or white flesh. Some green-fleshed melons are quite sweet, but most of the green- and white-fleshed melons have a less sweet, but very refreshing flavor.

Winter melons, (C. melo inodorus), have a hard rind which preserves freshness, include the well known casaba, crenshaw and honeydew types. They are usually quite large with smooth or netted rinds and can be deep green, bright yellow, beige and many shades in between. They come in just about any shape imaginable. The flesh can be green, white, pale yellow or orange and is usually incredibly sweet, but not very fragrant.

Honeydew melons are round to slightly oval shape and fruits at maturity of at least 1 kg (on average) or more. The flesh is usually pale green in color, while the smooth peel ranges from greenish to yellow. This fruit grows best in semiarid climates and is harvested based on maturity. Maturity can be hard to judge, but is based upon ground color ranging from greenish white to creamy yellow. Quality is also determined by the honeydew having a surface free of scars or defects. Also, a honeydew should feel heavy for its size and have a waxy surface.

Sprite melon is a kind of melon, similar to honeydew, which originates from Japan. Since the late 1990s, it has been cultivated in North Carolina as a specialty crop and has increased in popularity in recent years. A sprite melon has a round shape and is typically about the size of a grapefruit (less than 700 g on average). The flesh of a sprite melon is ivory in color and firm, while the peel ranges from ivory (unripe) to yellowish (ripe). As the melon reaches optimal ripeness, horizontal brown markings will appear near the stem. The firm, juicy, sweet flesh of the sprite melon is usually eaten for dessert and tends to resemble the flavor of both pears and honeydew, however it is much sweeter than either of these and can often consist of 18% sugar.

The use of a type of melon is depending on a customer's preference for taste, color and aroma of the melon flesh. There is thus a need for a white fleshed honeydew type of melon, especially white fleshed honeydew with unique flavor and aroma properties.

### SUMMARY OF THE INVENTION

In one aspect, a seed of a honeydew melon plant is provided, which plant produced honeydew melon fruits having a cream colored fruit flesh (referred to as ‘white honeydew melon’) and having an average fruit weight at edible maturity of at least 1000 grams.

Thus, seed of a honeydew melon plant are provided, which plant produced honeydew melon fruits having a cream colored fruit flesh and having an average fruit weight at edible maturity of at least 1000 grams.

In one aspect the said cream color has a color chart value of RHS157D.

In one aspect the plant produces fruits further comprising one or more characteristics selected from: the fruits have a non-netted rind and fruits having a rind thickness at medial of at least about 3 mm, preferably 3.2 mm, 3.3 mm, 3.4 mm or more.

In a further aspect the plant produces fruits further comprising one or more characteristics selected from: a) fruits have a strong fruit aroma wherein fruit aroma is selected from the categories: absent, faint and strong, b) fruits have a ‘tropical’ flavor, resembling a pineapple/coconut/banana flavor, c) fruits having an average fruit firmness after 14 days of storage at about 10° C. which is at least 95% of the firmness measured at harvest and d) the number of days from seeding to harvest are at least 10 days less than for variety Saturno.

In a further aspect of the invention a seed of melon variety NUN 01052 MEM is provided, wherein a representative sample of said seed has been deposited under Accession Number NCIMB ______.

Also a plant or plant part grown from these seeds are provided herein.

Thus, provided is a plant of melon variety NUN 01052 MEM, or a part thereof, wherein a representative sample of seed of said variety has been deposited under Accession Number NCIMB ______.

Also a fruit of melon variety NUN 01052 MEM is provided, or a plant part produced from the plant above. Thus, a white honeydew melon fruit produced on a plant grown from these seeds is provided.

Also progeny melon plants are provided, as well as methods for producing these, which produce white honeydew melon fruits according to the invention.

In one aspect, such progeny have all the physiological and morphological characteristics of melon variety NUN 01052 MEM when grown under the same environmental conditions.

In yet another embodiment of the invention, an Essentially Derived Variety of NUN 01052 MEM having one, two or three physiological and/or morphological characteristics which are different from those of NUN 01052 MEM and which otherwise has all the physiological and morphological characteristics of NUN 01052 MEM, wherein a representative sample of seed of variety NUN 01052 MEM has been deposited under Accession Number NCIMB ______.

Further, a vegetatively propagated plant of variety NUN 01052 MEM, or a part thereof, is provided having all the morphological and physiological characteristics of NUN 01052 MEM when grown under the same environmental conditions.

Also a plant part derived from variety NUN 01052 MEM is provided, wherein said plant part is selected from the group consisting of: harvested fruits or parts thereof, pollen, ovules, cells, leaves or parts thereof, petioles, shoots or parts thereof, stems or parts thereof, roots or parts thereof, cuttings, seeds, hypocotyl, cotyledon, flowers or parts thereof.

Also a method of producing a melon plant is provided, comprising crossing a melon plant according to the invention (as described above) with a second melon plant one or more times, and selecting progeny from said crossing.

Also a method of producing a melon plant is provided, comprising selfing a melon plant according to the invention (as described above) one or more times, and selecting progeny from said selfing.

Further, progeny of a melon plant according to the invention are provided which retain one or more essential characteristics, especially cream colored fruit flesh and flesh firmness after two weeks of storage at 10° C. remaining essentially as flesh firmness at harvest and/or progeny of melon variety NUN 01052 MEM obtained by further breeding with said variety, wherein said progeny produces honeydew melon fruits with cream colored fruit flesh and an average fruit weight at edible maturity of at least 1000 grams and optionally and flesh firmness after two weeks of storage at 10° C. remaining essentially as flesh firmness at harvest.

In one aspect progeny have all the physiological and morphological characteristics of melon variety NUN 01052 MEM when grown under the same environmental conditions.

Also an Essentially Derived Variety of NUN 01052 MEM is provided having one, two or three physiological and/or morphological characteristics which are different from those of NUN 01052 MEM and which otherwise has all the physiological and morphological characteristics of NUN 01052 MEM, wherein a representative sample of seed of variety NUN 01052 MEM has been deposited under Accession Number NCIMB ______.

A method of producing plants, or a part thereof, of variety NUN 01052 MEM comprising vegetative propagation of variety NUN 01052 MEM is also provided.

A vegetative propagated plant of variety NUN 01052 MEM, or a part thereof, having all the morphological and physiological characteristics of NUN 01052 MEM when grown under the same environmental conditions is also provided herein.

A plant part derived from variety NUN 01052 MEM is provided, wherein said plant part are harvested fruit or parts thereof, pollen, cells, leaves or parts thereof, petioles, shoots or parts thereof, stems or parts thereof, roots or parts thereof, cuttings, or flowers or parts thereof.

### DEFINITIONS

“Melon” or “muskmelon” refers herein to plants of the species Cucumis melo. “Cultivated melon” refers to plants of Cucumis melo i.e. varieties, breeding lines or cultivars of the species C. melo, cultivated by humans and having good agronomic characteristics; preferably such plants are not “wild plants”, i.e. plants which generally have much poorer yields and poorer agronomic characteristics than cultivated plants and e.g. grow naturally in wild populations. “Wild plants” include for example ecotypes, PI (Plant Introduction) lines, landraces or wild accessions or wild relatives of a species.

“Honeydew melon” refers herein to a type of C. melo inodorus, wherein the fruits have a round to slightly oval shape, and its flesh is green in color (ranging from pale green to deep green), while the smooth (non-ridged) skin (or rind) ranges from cream to yellow. An examples is variety Saturno (Seminis Vegetable Seeds).

“White honeydew” melon refers herein to a honeydew melon which comprises fruits with cream-colored flesh instead of green colored flesh and which further comprises one or more distinguishing characteristics (as described herein), for example characteristics selected from the group: fruits having an average fruit weight of at least about 1000 grams or more, fruits having a non-netted rind, fruits having a strong fruit aroma, fruits having a “somewhat spicy” fruit flavor, and fruits having a rind thickness at medial of at least about 3 mm, preferably 3.2 mm, 3.3 mm, 3.4 mm or more and fruits having good storage properties, especially fruit texture (firmness) after two weeks of storage at about 10° C. is essentially the same as at harvest, as determinable by using a penetrometer.

“Non-ridged” skin or rind refers to the absence of ridges or fissures.

“Non-netted” skin or rind refers to the absence of reticulate markings called ‘netting’ on the skin.

“UPOV descriptors” are the plant variety descriptors described for Muskmelon in the “Guidelines for the Conduct of Tests for Distinctness, Uniformity and Stability, TG/184/3 (Geneva 2001), as published by UPOV (International Union for the Protection of New Varieties and Plants, available on the world wide web at upov.int) and which can be downloaded from the world wide web at www.upov.int/en/publications/tg_rom/tg_index.html and is herein incorporated by reference in its entirety.

As used herein, the term “plant” includes the whole plant or any parts or derivatives thereof, preferably having the same genetic makeup as the plant from which it is obtained, such as plant organs (e.g. harvested or non-harvested fruits), plant cells, plant protoplasts, plant cell tissue cultures from which whole plants can be regenerated, plant calli, plant cell clumps, plant transplants, seedlings, hypocotyl, cotyledon, plant cells that are intact in plants, plant clones or micropropagations, or parts of plants (e.g. harvested tissues or organs), such as plant cuttings, vegetative propagations, embryos, pollen, ovules, fruits, flowers, leaves, seeds, clonally propagated plants, roots, stems, root tips, grafts, parts of any of these and the like. Also any developmental stage is included, such as seedlings, cuttings prior or after rooting, mature plants or leaves.

“Harvested plant material” refers herein to plant parts (e.g. fruits detached from the whole plant) which have been collected for further storage and/or further use.

“Harvested seeds” refers to seeds harvested from a line or variety, e.g. produced after self-fertilization or cross-fertilization and collected.

As used herein, the term “variety” or “cultivar” means a plant grouping within a single botanical taxon of the lowest known rank, which grouping, irrespective of whether the conditions for the grant of a breeder's right are fully met, can be defined by the expression of the characteristics resulting from a given genotype or combination of genotypes, distinguished from any other plant grouping by the expression of at least one of the said characteristics and considered as a unit with regard to its suitability for being propagated unchanged.

A variety is referred to as an “Essentially Derived Variety” (EDV) i.e., shall be deemed to be essentially derived from another variety, “the initial variety” when (i) it is predominantly derived from the initial variety, or from a variety that is itself predominantly derived from the initial variety, while retaining the expression of the essential characteristics that result from the genotype or combination of genotypes of the initial variety; (ii) it is clearly distinguishable from the initial variety; and (iii) except for the differences which result from the act of derivation, it conforms to the initial variety in the expression of the essential characteristics that result from the genotype or combination of genotypes of the initial variety. Thus, an EDV may be obtained for example by the selection of a natural or induced mutant, or of a somaclonal variant, the selection of a variant individual from plants of the initial variety, backcrossing, or transformation by genetic engineering.

“Plant line” is for example a breeding line which can be used to develop one or more varieties.

“Hybrid variety” or “F1 hybrid” refers to the seeds harvested from crossing two inbred (nearly homozygous) parental lines. For example, the female parent is pollinated with pollen of the male parent to produce hybrid (F1) seeds on the female parent.

“Average” refers herein to the arithmetic mean.

“Locus” (plural loci) refers to the specific location of a gene or DNA sequence on a chromosome. A locus may confer a specific trait.

“Allele” refers to one or more alternative forms of a gene locus. All of these loci relate to one trait. Sometimes, different alleles can result in different observable phenotypic traits, such as different pigmentation. However, many variations at the genetic level result in little or no observable variation. If a multicellular organism has two sets of chromosomes, i.e. diploid, these chromosomes are referred to as homologous chromosomes. Diploid organisms have one copy of each gene (and therefore one allele) on each chromosome. If both alleles are the same, they are homozygotes. If the alleles are different, they are heterozygotes.

“Genotype” refers to the genetic composition of a cell or organism.

“Maturity” refers to the fruit developmental stage when the fruit has fully developed (reached its final size), begins to ripen and undergoes ripening, during which fruits can be divided into maturity stages 1, 2 and 3. Thereafter, fruits become overripe.

“Harvest maturity” or ““near minimal commercial maturity” refers to stage 1 maturity (“mature unripe”) when fruits contain an SSC (soluble solids content) of about 10%.

“Edible maturity” or “commercial maturity” refers to stage 2 maturity (“mature ripening” stage).

“Full maturity” refers to stage 3 maturity (“ripe” stage; abscission zone is forming).

“Abscission zone” is the layer or zone separating the fruit from the peduncle.

“Cavity” or “seed cavity” is the center of the fruit containing the maternal tissues and seeds.

“Flavor” refers to the sensory impression of a food or other substance, especially a melon fruit or fruit part (fruit flesh) and is determined mainly by the chemical senses of taste and smell. Flavor is influenced by texture properties and by volatile and/or non-volatile chemical components (organic acids, lipids, carbohydrates, etc.).

“Aroma” refers to smell (or odor) characteristics of melon fruits or fruit parts (fruit flesh).

Royal Horticultural Society (RHS) color chart is the standard reference for plant color identification.

“Trained Sensory Panel” is a group of people that can determine the flavor, aroma and/or texture properties of melon fruits. Such a qualified trained sensory taste panel can, e.g., be formed by companies like Scientific Certification Systems Inc., 2000 Powell Street, Suite 600, Emeryville, Calif., 94608. Generally, a qualified trained sensory taste panel contains at least 7, 8, 9, 10, 12, 14, 16, 18, 20 or even more panelists. At least 4, 5, 6 or even more panelists are generally present in a test session. In another embodiment, at least 80%, at least 90%, all panelists are trained in Calibration solutions for flavors, aromas and/or textures, e.g. sweet, sour, bitter, juiciness, hardness, graininess, mild to somewhat spicy to very spicy (indicating the strength of the flavor, especially ‘muskiness’), tropical, etc. before tasting. Sensations can be rated on predefined scales for texture (e.g. from mushy to firm), sweetness (e.g. from not sweet to very sweet), predefined flavor categories or combinations of these (e.g. melon-, banana-, strawberry-, apple-, pear-, pineapple-, lemon-, mango-, coconut-flavor, or others). Trained sensory panels can also rate the overall sweet taste acceptance, texture acceptance, aroma acceptance and overall quality acceptance on a defined scale e.g. 0-15 whereby 15 is the highest acceptance rating.

The term “traditional breeding techniques” encompasses herein crossing, selfing, selection, double haploid production, embryo rescue, protoplast fusion, marker assisted selection, mutation breeding etc. as known to the breeder (i.e. methods other than genetic modification/transformation/transgenic methods), by which, for example, a genetically heritable trait can be transferred from one melon line or variety to another.

“Backcrossing” is a traditional breeding technique used to introduce a trait into a plant line or variety. The plant containing the trait is called the donor plant and the plant into which the trait is transferred is called the recurrent parent. An initial cross is made between the donor parent and the recurrent parent to produce progeny plants. Progeny plants which have the trait are then crossed to the recurrent parent. After several generations of backcrossing and/or selfing the recurrent parent comprises the trait of the donor. The plant generated in this way may be referred to as a “single trait converted plant”.

### DRAWINGS

The drawings described herein are for illustration purposes only and are not intended to limit the scope of the present teachings in any way.

FIG. 1 is a photograph of three melon fruits at full maturity of each Saturno, NUN 01052 MEM, and Sprite.

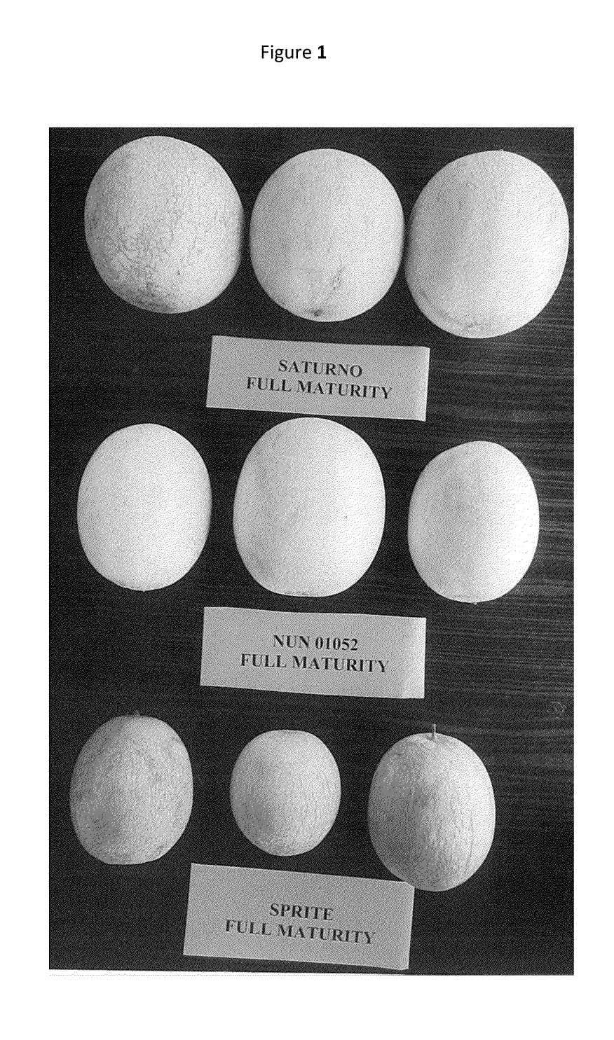

FIG. 2 is a photograph of a sliced-in-half melon fruit of Saturno, NUN 01052 MEM, and Sprite.

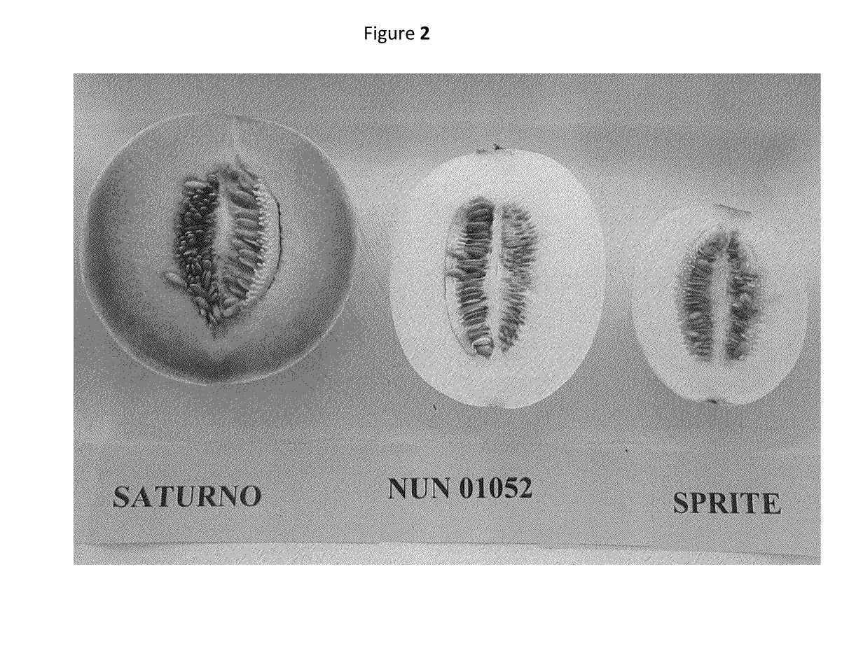

FIG. 3 is a photograph of 1 gram of seeds of Sprite, NUN 01052 MEM, and Saturno. The scale is in centimeters.

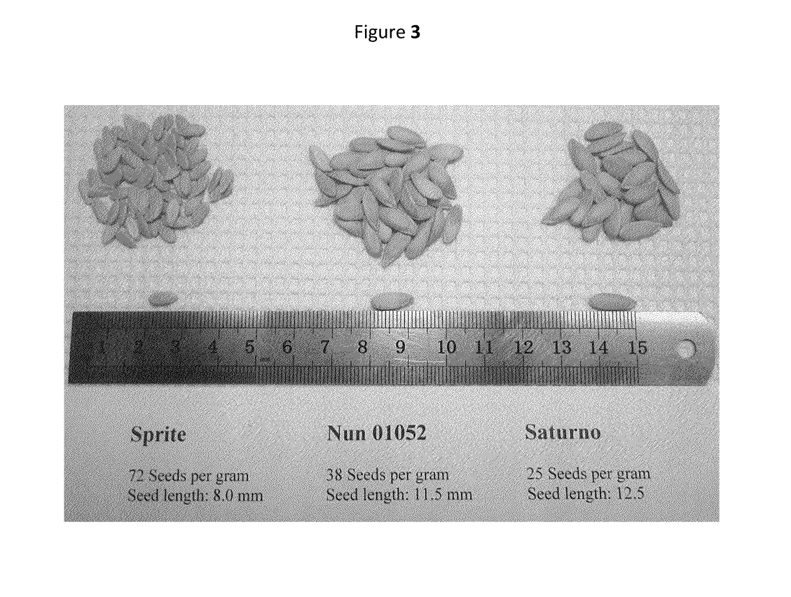

### DETAILED DESCRIPTION

The invention relates to a honeydew melon type, which however has cream-colored flesh, similar to the Japanese Sprite type, instead of the, for honeydew types typical, green or greenish flesh at maturity and throughout ripening (i.e. at harvest maturity, edible maturity and full maturity). The flesh color of the honeydew melons according to the invention has, on the Royal Horticultural Society (RHS) color chart, a value of RHS157D, while the flesh color of Sprite has a value of RHS155C. In addition, the new cream-fleshed honeydew type (also referred herein to as ‘white honeydew’) also has a unique flavor and aroma, as well as excellent storage properties. The white honeydew fruit is not netted (and non-ridged) and produces fruits which are significantly larger than Sprite, namely an average fruit weight of at least about 1000 g or more.

Thus, the white honeydew provided herein differs from Sprite type melons and Oriental melons in one or more characteristics (referred herein to as “distinguishing characteristics” or “distinguishing morphological and/or physiological characteristics”) selected from: i) earliness (requiring significantly fewer days from seeding to harvest maturity, especially plants requiring significantly fewer days from seeding to harvest, reaching maturity at least about 5, 6, 7, 8 or more days earlier than oriental melons such as Sprite), ii) average fruit size (fruits being significantly larger, having an average fruit length and/or fruit diameter of at least about 1 cm, or at least about 2 cm larger, and having a significantly higher fruit weight, e.g. weighing on average at least about 1000 g or more), iii) having a smooth fruit surface (without netting), iv) having a strong fruit aroma (selected from the categories: absent, faint and strong), v) having a ‘somewhat spicy fruit flavor’ (selected from the categories: mild, somewhat spicy and very spicy), vi) having on average a significantly larger seed cavity, e.g. the seed cavity being at 10, 15, or 20 millimeters wider), vii) having on average significantly larger seeds, seeds being on average at least about 1, 2 or 3 millimeters longer, viii) having on average significantly more seeds per fruit, such as at least about 100, 150, or 200 more seeds per fruit, ix) fruits having on average significantly thicker rind at medial, such as the rind being at least about 0.5, 0.6, 0.7, 0.8, 0.9 or 1 mm thicker, x) having a rind color at full maturity which is significantly lighter (e.g. rind color being cream-yellow rather than yellow, and xi) having a blossom scar that is obscure rather than conspicuous, xii) having a weak strength of attachment of peduncle at maturity compared to very strong for Sprite on a scale of very weak, weak, medium, strong, very strong as defined in UPOV Breeder's reference.

As mentioned, the white honeydew melon type provided herein differs from known honeydew melons in having a cream colored fruit flesh at maturity and throughout ripening, i.e. at harvest maturity, edible maturity and full maturity.

In addition, in one embodiment of the invention, the white honeydew provided herein differs from known honeydew melons (such as Saturno) in one or more characteristics (referred herein to as “distinguishing characteristics” or “distinguishing morphological and/or physiological characteristics”) selected from: a) having an early maturity (days from seeding to harvest) of at least about 10 days earlier than Saturno and at least about 4 or 5 days earlier than Sprite, when grown under the same conditions; depending on growing conditions maturity is reached in less than 70 days from seeding, preferably in less than 69, 68, 67 or 66 days; b) having a strong fruit aroma (selected from the categories: absent, faint and strong); c) having a ‘somewhat spicy fruit flavor’ (selected from the categories: ‘mild’, ‘somewhat spicy’ and ‘very spicy’, which indicates the flavor strength, especially the muskiness); d) having a ‘tropical’ flavor, resembling a pineapple/coconut/banana flavor; e) fruits having on average significantly thicker rind at medial, such as the rind being at least about 0.2, 0.3, 0.4, 0.5 or 0.6 mm thicker; f) having significantly improved post-harvest storage at full ripeness, such as remaining essentially free of external blemishes and having good eating quality until at least about 14, 15, 16, 17, 18, 19, 20, 21, 22, 23, 24, or more, days storage at about 10° C. or below (e.g. between about 7° C. and about 10° C., preferably at relative humidity of at least about 85%, preferably at least about 90% or 95%) and/or having an average fruit firmness after at least about 14, 15, 16, 17, 18, 19, 20, 21, 22, 23, 24, or more, days storage at about 10° C. which is essentially the same as the fruit firmness at harvest as determinable using a penetrometer (essentially the same meaning that the firmness measured after the period of storage is at least 95%, 96%, 97%, 98% or 99% of the firmness measured at harvest), g) the fruits of the plants of the invention having a medium rate of change of skin color from maturity to over maturity compared to an absent/very slow rate for Saturno, on a scale of absent/very slow, slow, medium, fast as defined in UPOV Breeder's reference.

It is understood that “significant” differences refer to statistically significant differences, when comparing the characteristic between two plant lines or varieties when grown under the same conditions. Preferably at least about 10, 15, 20 or more plants per line or variety are grown under the same conditions and characteristics are measured on at least about 10, 15, 20 or more randomly selected plant or plant parts to obtain averages. Thus, physiological and morphological characteristics or traits are commonly evaluated at a significance level of 1%, 5% or 10%, when measured in plants grown under the same environmental conditions.

In a further embodiment the invention provides for a hybrid variety of Cucumis melo called NUN 01052 MEM. The invention also provides for a plurality of seeds of the new honeydew type and of the new variety, plants produced from growing the seeds and also plant parts obtainable from the grown plant, such as fruits or parts thereof or pollen, cells, leaves or parts thereof, petioles, shoots or parts thereof, stems or parts thereof, roots or parts thereof, cuttings, flowers, and vegetative reproductions of the white honeydew type or of the new variety NUN 01052 MEM, and progeny of any of these. Especially, progeny retaining one or more of the “distinguishing characteristics” referred to above and elsewhere herein, are encompassed herein. The embodiments described herein for NUN 01052 MEM are understood to equally apply to the new white honeydew type in general and to not be limited to variety NUN 01052 MEM. One or more (or all) of the distinguishing characteristics can be transferred to other honeydew melons by crossing and selection of phenotype of one or more of the distinguishing characteristics in the progeny. Thus, for example the cream color and the flesh firmness after storage can be transferred to other honeydew melons.

Thus, in one aspect, the invention provides seeds of a honeydew melon plant, wherein the fruits of said melon plant have cream colored flesh at maturity and during ripening (i.e. at harvest maturity, edible maturity and/or full maturity) and wherein the plant further has one or more distinguishing characteristics selected from the group: i) earliness (requiring significantly fewer days from seeding to harvest maturity, especially plants requiring significantly fewer days from seeding to harvest, reaching maturity at least about 5, 6, 7, 8 or more days earlier than oriental melons such as Sprite), ii) average fruit size (fruits being significantly larger than oriental melons such as Sprite, having an average fruit length and/or fruit diameter of at least about 1 cm, or at least about 2 cm larger, and having a significantly higher fruit weight, e.g. weighing on average at least about 1000 g or more), iii) having a smooth fruit surface without netting, iv) having a strong fruit aroma (selected from the categories: absent, faint and strong), v) having a ‘somewhat spicy fruit flavor’ (selected from the categories: mild, somewhat spicy and very spicy, which refers to the flavor strength, especially muskiness), vi) having on average a significantly larger seed cavity, e.g. the seed cavity being at 10, 15, or 20 millimeters wider than oriental melons such as Sprite), vii) having on average significantly larger seeds than oriental melons such as Sprite, seeds being on average at least about 1, 2 or 3 millimeters longer, viii) having on average significantly more seeds per fruit than oriental melons such as Sprite, such as at least about 100, 150, or 200 more seeds per fruit, ix) fruits having on average significantly thicker rind at medial than oriental melons such as Sprite, such as the rind being at least about 0.5, 0.6, 0.7, 0.8, 0.9 or 1 mm thicker, x) having a rind color at full maturity which is significantly lighter than in oriental melons such as Sprite (e.g. rind color being cream-yellow rather than yellow, and xi) having a blossom scar that is obscure rather than conspicuous.

In a further aspect, the invention provides seeds of a honeydew melon plant, wherein the fruits of said melon plant have cream colored flesh at maturity and during ripening (at harvest maturity, edible maturity and/or full maturity) and wherein the plant further has one or more distinguishing characteristics selected from the group: fruits reaching maturity at least about 10 days earlier than variety Saturno; fruits having an average fruit weight of at least about 1000 grams or more; fruits having a smooth fruit surface (no netting); fruits having a strong fruit aroma; fruits having a somewhat spicy fruit flavor, and fruits having a rind thickness at medial of at least about 3 mm, preferably 3.2 mm, 3.3 mm, 3.4 mm or more, and fruits having an average fruit firmness after at least about 14, 15, 16, 17, 18, 19, 20, 21, 22, 23, 24, or more, days storage at about 10° C. which is essentially the same as the fruit firmness at harvest as determinable using a penetrometer (essentially the same meaning that the firmness measured after the period of storage is at least 95%, 96%, 97%, 98% or 99% of the firmness measured at harvest).

In another aspect, the invention provides seeds of a honeydew melon plant, wherein the fruits of said melon plant have cream colored flesh at maturity and during ripening (at harvest maturity, edible maturity and/or full maturity) and wherein the plant further has one or more distinguishing characteristics selected from the group: a) having a strong fruit aroma (selected from the categories: absent, faint and strong), b) having a ‘somewhat spicy fruit flavor’ (selected from the categories: mild, somewhat spicy and very spicy), c) having a ‘tropical’ flavor, resembling a pineapple/coconut/banana flavor, d) fruits having on average significantly thicker rind at medial, such as the rind being at least about 0.2, 0.3, 0.4, 0.5 or 0.6 mm thicker than the rind of honeydew melons such as Saturno and/or than the rind of oriental melons such as Sprite, i.e. the rind being at least about 3.2 mm, 3.3 mm, 3.4 mm, 3.5 mm or 3.6 mm thick at medial; e) having significantly improved post-harvest storage at full ripeness, such as remaining essentially free of external blemishes and having good eating quality until at least about 14, 15, 16, 17, 18, 19, 20, 21, 22, 23, 24, or more, days storage at about 10° C. or below (e.g. between about 7° C. and 10° C.; preferably at a relative humidity of at least about 85%, preferably at least about 90% or 95% RH); f) fruits having an average fruit firmness after at least about 14, 15, 16, 17, 18, 19, 20, 21, 22, 23, 24, or more, days storage at about 10° C. (preferably at a relative humidity, RH, of at least about 85%, preferably at least about 90% or 95% RH) which is essentially the same as the fruit firmness at harvest as determinable using a penetrometer (essentially the same meaning that the firmness measured after the period of storage is at least 95%, 96%, 97%, 98% or 99% of the firmness measured at harvest).

In another aspect, the invention provides seeds from which a melon plant having the above mentioned characteristics can be grown. One or more of these characteristics are obtainable from or can be derived from melon variety NUN01052MEM, a representative sample of seeds of said variety was deposited under the Budapest Treaty, with Accession number NCIMB ______, for example by crossing a melon plant with variety NUN01052MEM or with a progeny plant of variety NUN01052MEM (e.g. a plant obtained by selfing variety NUN01052MEM one or more times) and by selecting amongst progeny plants derived from said cross those plants which comprise one or more of the distinguishing characteristics.

Also honeydew fruits obtainable from such plants and having cream colored flesh at maturity and during ripening (at harvest maturity, edible maturity and/or full maturity) and one or more of the distinguishing characteristics are encompassed herein.

In another aspect, the invention provides seeds of melon variety designated NUN 01052 MEM wherein a representative sample of seeds of said variety was deposited under the Budapest Treaty, with Accession number NCIMB ______.

In another aspect, the invention provides for a melon plant of melon variety NUN 01052 MEM, a representative sample of seed from said variety has been deposited under the Budapest Treaty, with Accession number NCIMB ______.

In other aspects, the invention provides for a fruit of melon variety NUN 01052 MEM, or a plant part, such as pollen, flowers, shoots or cuttings of variety NUN 01052 MEM or parts thereof.

In other aspects, the invention provides for progeny of variety NUN 01052 such as progeny obtained by selfing NUN 01052 MEM one or more times and/or cross-pollinating NUN 01052 with another melon plant or variety one or more times. In particular, the invention provides for progeny that retain all the essential morphological and physiological characteristics of NUN 01052 MEM or that retain the cream-colored flesh at fruit maturity and ripening and/or that retain one or more of the distinguishing characteristics of the white honeydew type described further above and when grown under the same environmental conditions. In another aspect, the invention provides for vegetative reproductions of the variety are provided and essentially derived varieties (EDVs) of NUN 01052 MEM.

The present invention further provides a new honeydew melon variety NUN 01052 MEM, the fruits of which having a cream rind color at edible maturity, a cream-yellow rind color at full maturity and a cream flesh color at edible maturity and throughout ripening. The plants of NUN 01052 MEM are most similar to Saturno which is also a Honeydew type melon. The fruit color of NUN 01052 MEM is most similar to the fruit color of Sprite, an oriental variety similar to honeydew, which originates from Japan. However, NUN 01052 MEM differs in a number of characteristics and can easily be distinguished from both Sprite and Saturno when grown under the same environmental conditions, see Table 1.

Firstly, the average number of days to maturity, i.e. from seeding to harvest is significantly shorter for NUN 01052 MEM compared to both Sprite and Saturno. Also the average fruit size, both the length and diameter of NUN 01052 is bigger than that of Sprite, which results in a significant higher average weight per melon fruit of NUN 01052 MEM compared to Sprite. The melon fruit of variety Saturno have a slightly higher weight than NUN 01052 MEM.

Melon fruit of NUN 01052 MEM have an obscure blossom scar while both Sprite and Saturno have a conspicuous blossom scar. The blossom scar of Sprite being more conspicuous. In addition, melon fruit of NUN 01052 MEM has no rind net (no netting) while Sprite and Saturno have netted rinds. Saturno has an intermediate sparse rind netting while Sprite has a sparse rind netting. The color of the netting at edible maturity and at full maturity for both Sprite and Saturno is bronze.

Furthermore, the most obvious difference between NUN 01052 MEM and Saturno is the fruit flesh color. The flesh of NUN 01052 being cream color (RHS 157D) throughout the fruit (near rind, center and near cavity), while that of Saturno is green (RHS 142). Also the fruit flesh of NUN 01052 MEM has a strong aroma (on a scale of absent-mild-strong) and a ‘somewhat spicy’ flavor (on a scale of mild-somewhat spicy-very spicy), while in both Sprite no aroma is present and in Saturno only a faint aroma is present at edible maturity and flavor is mild for both Sprite and Saturno fruits at edible maturity.

The seed cavity of NUN 01052 MEM is significantly larger (at least about 90 mm long) than that of Sprite and Saturno and also the average number of seeds per fruit of NUN 01052 MEM is significantly higher (at least 500 seeds, preferably at least 550 seeds per fruit) than in Sprite and Saturno. Seeds of NUN 01052 MEM are significantly longer than those of Sprite, those of NUN 01052 MEM being on average at least about 10 mm long, preferably at least about 11 mm or 11.5 mm long.

As discussed herein, one of the main differences between NUN 01052 MEM and Saturno (the most comparable honeydew variety) is the color of the flesh of the melon which has a creme color in NUN 01052 MEM (RHS 157D throughout) while flesh color is green in Saturno (RHS color chart value of 142A near cavity, 142B near center and 142D near rind).

The morphological and/or physiological differences between white honeydew plants according to the invention, such as NUN 01052 MEM or progeny thereof, and other known varieties can easily be established by growing NUN 01052 MEM next to the other varieties (in the same field, under the same environmental conditions), preferably in several locations which are suitable for honeydew melon cultivation, and measuring morphological and/or physiological characteristics of a number of plants (e.g., to calculate an average value and to determine the variation range/uniformity within the variety). For example, trials can be carried out in Acampo Calif., USA (N 38 degrees 07′261″/W 121 degrees 18′ 807″, USA, whereby e.g. type of melon, area of best adaption in USA, days from seeding to harvest, plant fertility, plant habit, leaf shape, leaf lobes, leaf color, leaf length, width and surface, fruit length, fruit diameter, fruit weight, fruit shape, fruit surface, blossom scar, rib presence and number of ribs per fruit, rib width, rib surface, suture depth, suture surface, shipping quality, fruit abscission, rind net presence/distribution/coarseness/interlacing/interstices, rind texture, rind texture thickness, rind color at edible maturity, mottling color, net color, furrow (suture), rind color at full maturity, mottling color, net color, furrow (suture), flesh color near cavity, flesh color in center, flesh color near rind, % soluble solids, flesh aroma, flesh flavor, seed cavity length, seed cavity width, seed cavity shape, number of seeds per fruit, weight per 1,000 seeds, disease resistances and/or insect resistances can be measured and directly compared.

Morphological and physiological characteristics (and distinguishing characteristics) of white honeydew melon, such as NUN 01052 MEM, are provided in the Examples, in Table 1. Encompassed herein are also plants derivable from white honeydew melons, such as derivable from NUN 01052 MEM (e.g. by selfings and/or crossing and/or backcrossing with NUN 01052 MEM and/or progeny thereof) comprising all the physiological and morphological characteristics of NUN 01052 listed in Table 1 as determined at the 5% significance level when grown under the same environmental conditions and/or comprising one or more of the distinguishing characteristics as determined at the 5% significance level when grown under the same environmental conditions.

Also at-harvest and/or post-harvest characteristics of fruits can be compared, such as cold storage holding quality (browning), post-harvest rind firmness and/or flesh firmness, and juiciness can be measured using known methods. The white-flesh honeydew according to the invention, e.g. NUN 01052 MEM or progeny, has excellent storage properties after harvest and/or at full ripeness, e.g. fruits remaining essentially free of external blemishes and having good eating quality until at least about 14, 15, 16, 17, 18, 19, 20, 21, 22, 23, 24, or more, days storage at about 10° C. or below (e.g. between about 7° C. and 10° C.; preferably at a relative humidity of at least about 85%, preferably at least about 90% or 95% RH). Especially, fruits have an average fruit firmness after at least about 14, 15, 16, 17, 18, 19, 20, 21, 22, 23, 24, or more, days storage at about 10° C. (preferably at a relative humidity, RH, of at least about 85%, preferably at least about 90% or 95% RH) which is essentially the same as the fruit firmness at harvest as determinable using a penetrometer (essentially the same meaning that the firmness measured after the period of storage is at least 95%, 96%, 97%, 98% or 99% of the firmness measured at harvest).

As can be seen in the Examples, NUN01052 ME has excellent storage properties at 10 degrees Celsius. Fruit flesh texture (firmness), as measured using a penetrometer, is essentially as firm after 2 weeks of storage at 10 degrees Celsius as it is at harvest, while in comparative green-fleshed varieties average fruit flesh firmness is significantly decreased after two weeks compared to harvest (in varieties Santa Fe and Jade Delight, for example, fruit flesh firmness is only 67% and 54% of the firmness at harvest, respectively). Also the sugar and organic acid levels of NUN1052ME are very good two weeks after harvest, with fruits having higher average glucose, fructose and sucrose levels after two weeks of storage at 10 degrees Celsius compared to the levels at harvest. Likewise, citric acid, isocitric acid, malic acid and succinic acid levels were increased compared to harvest. Thus, the white-flesh honeydew according to the invention, e.g. NUN 01052 MEM or progeny, has excellent storage properties, with fruits having an average fruit firmness after at least about 14, 15, 16, 17, 18, 19, 20, 21, 22, 23, 24, or more, days storage at about 10° C. (preferably at a relative humidity, RH, of at least about 85%, preferably at least about 90% or 95% RH) which is essentially the same as the fruit firmness at harvest as determinable using a penetrometer (essentially the same meaning that the firmness measured after the period of storage is at least 95%, 96%, 97%, 98% or 99% of the firmness measured at harvest).

Flesh firmness can for example be measured using a penetrometer, e.g. by inserting a probe into the fruit flesh and determining the insertion force, or other methods.

The morphological and/or physiological characteristics may vary somewhat with variation in the environment (such as temperature, light intensity, day length, humidity, soil, fertilizer use), which is why a comparison under the same environmental conditions is preferred. Colors can best be measured against The Munsell Book of Color (Munsell Color Macbeth Division of Kollmorgan Instruments Corporation) or using the Royal Horticultural Society Chart (http://www.rhs.org.uk/Plants/RHS-Publications/RHS-colour-charts).

In one embodiment, the fruits of white honeydew melons according to the invention, such as NUN01052 and progeny thereof, has good sensory attributes and especially and very good texture acceptance and overall eating quality acceptance at harvest. Especially, fruits according to the invention have an overall texture acceptance at harvest of at least 12.3 and/or an overall quality acceptance of at least 12.6 on a scale of 1-3=unacceptable, 4-6=below average, 7-9=average, 10-12=good, and 13-15=excellent, as determined by a trained sensory panel.

Seeds of honeydew variety NUN 01052 MEM are provided herein, wherein a representative sample of said seeds (2500 seeds) has been deposited, under the Budapest Treaty, with Accession Number NCIMB ______.

Seeds of NUN 01052 MEM are obtainable by crossing the male parent with the female parent and harvesting the seeds produced on the female parent. The resultant NUN 01052 MEM seeds can be grown to produce NUN 01052 MEM plants. In one embodiment a plurality of NUN 01052 MEM seeds are packaged into small and/or large containers (e.g., bags, cartons, cans, etc.). The seeds may be treated with various compounds, such as seed coatings or fungicides or insecticides.

Also provided are plants of melon variety NUN 01052 MEM, or a fruit or other plant part thereof, produced from seeds, wherein a representative sample of said seeds has been deposited under the Budapest Treaty, with Accession Number NCIMB ______. Plants of NUN 01052 MEM can be produced by seeding directly in the ground (e.g., field) or by germinating the seeds in controlled environment conditions (e.g., greenhouses) and then transplanting the seedlings into the field. For example by sowing the seed into prepared seed beds where they will remain for the entire production of the crop. Alternatively, the melon seed may be planted through a black plastic mulch. The dark plastic will absorb heat from the sun, warming the soil early. It will also help to conserve moisture during the growing season, controls weeds and makes harvesting easier and cleaner. See for example www.anrcatalog.ucdavis.edu for cultivation, harvesting, handling and postharvest methods commonly used.

Parts of NUN 01052 MEM encompass any cells, tissues, organs obtainable from the seedlings or plants, such as but not limited to: melon fruits or parts thereof, cuttings, hypocotyl, cotyledon, pollen and the like. Such parts can be stored and/or processed further. Encompassed are therefore also food or feed products comprising one or more of such parts, such as canned melon fruit from NUN 01052 MEM or from progeny thereof, or from a derived variety, such as an EDV.

In a preferred embodiment, the invention provides for melon fruits of variety NUN 01052 MEM, or a part of the fruit. In another embodiment, the invention provides for a container comprising or consisting of a plurality of harvested melon fruits of NUN 01052 MEM, or progeny thereof, or a derived variety, such as an EDV.

In yet a further embodiment, the invention provides for a method of producing a new melon plant, in particular a white honeydew melon plant according to the invention. The method comprises crossing a white honeydew plant, such as NUN 01052 MEM or an EDV thereof or a progeny plant thereof, either as male or as female parent, with a second melon plant (or a wild relative of melon) one or more times, and/or selfing a white honeydew melon plant according to the invention, such as NUN 01052 MEM or an EDV thereof or a progeny plant thereof, one or more times, and selecting progeny from said crossing and/or selfing.

Progeny are either the generation (seeds) produced from the first cross (F1) or selfing (S1), or any further generation produced by crossing and/or selfing (F2, F3, etc.) and/or backcrossing (BC1, BC2, etc.) one or more selected plants of the F1 and/or S1 and/or BC1 generation (or plants of any further generation, e.g. the F2) with another melon plant (and/or with a wild relative of melon). Progeny may have all the physiological and morphological characteristics of melon variety NUN 01052 MEM when grown under the same environmental conditions and/or progeny may have (be selected for having) one or more of the distinguishing characteristics of white honeydew melons of the invention. Using common breeding methods such as backcrossing or recurrent selection, one or more specific characteristics may be introduced into white honeydew melon plants, e.g. into NUN 01052 MEM, to provide an EDV of NUN 01052 MEM.

The invention provides for methods of producing varieties which retain all the morphological and physiological characteristics of NUN 01052 MEM and/or which retain one or more (or all) the distinguishing characteristics of white honeydew melons. The invention provides also for methods of producing EDVs (Essentially Derived Varieties) of NUN 01052 MEM which may differ from NUN 01052 MEM in one, two, three or more morphological and/or physiological characteristics, but which are still genetically closely related to NUN 01052 MEM. The relatedness can, for example be determined by fingerprinting techniques (e.g., making use of isozyme markers and/or molecular markers such as SNP markers, AFLP markers, microsatellites, minisatellites, RAPD markers, RFLP markers and others). An plant is “closely related” to NUN 01052 MEM if its DNA fingerprint is at least 80%, 90%, 95% or 98% identical to the fingerprint of NUN 01052 MEM. In a preferred embodiment AFLP markers are used for DNA fingerprinting (Vos et al. 1995, Nucleic Acid Research 23: 4407-4414). A closely related plant may have a Jaccard's Similarity index of at least about 0.8, preferably at least about 0.9, 0.95, 0.98 or more (Pisanu et al. ISHS 2004, Acta Hort. 660).

By crossing and/or selfing also (one or more) single traits may be introduced into a white honeydew melon, such as NUN 01052 MEM (e.g., using backcrossing breeding schemes), while retaining the remaining morphological and physiological characteristics of NUN 01052 MEM and/or while retaining one or more distinguishing characteristics. A single trait converted plant may thereby be produced. For example, disease resistance genes may be introduced, genes responsible for one or more quality traits, yield, etc. Both single genes (dominant or recessive) and one or more QTLs (quantitative trait loci) may be transferred into NUN 01052 MEM by breeding with NUN 01052 MEM.

Any pest or disease resistance genes may be introduced into a white honeydew plant according to the invention, such as NUN 01052 MEM, progeny thereof or into an EDV of NUN 01052 MEM. Resistance to one or more of the following diseases is preferably introduced into plants of the invention: Bacterial Wilt, Root Rot, Crown Blight, Melon Rust, Powdery Mildew, Verticillum Wilt, Sulphur Burn, Scab, Watermelon Mosaic, Downy Mildew, Fusarium oxysporum f.sp. melonis (Fom) race 0, Fusarium oxysporum f.sp. melonis (Fom) race 1, Fusarium oxysporum f.sp. melonis (Fom) race 2, Fusarium oxysporum f.sp. melonis (Fom) race 1-2, Fusarium Wilt R2, Root Knot (Nematode), Anthracnose, Cucumber Mosiac, and Squash Mosaic.

Resistance to one or more of the following pests is preferably present or introduced into plants of the invention: Aphid resistance, Pickle Worm, Darkling Ground Beetle, Banded Cucumber Beetle, Mite, Western Spotted Cucumber Beetle, Melon Leafhopper, Melon Worm, Western Striped Cucumber Beetle or Melon Leafminer. Other resistance genes, against pathogenic viruses, fungi, bacteria or pests may also be introduced.

Thus, invention also provides a method for developing an melon plant in a melon breeding program, using a melon plant of the invention, or its parts as a source of plant breeding material. Suitable plant breeding techniques are recurrent selection, backcrossing, pedigree breeding, mass selection, mutation breeding and/or genetic marker enhanced selection. For example, in one aspect, the method comprises crossing NUN 01052 MEM or progeny thereof, or an EDV thereof, with a different melon plant, and wherein one or more offspring of the crossing are subject to one or more plant breeding techniques selected from the group consisting of recurrent selection, backcrossing, pedigree breeding, mass selection, mutation breeding and genetic marker enhanced selection (see e.g. Martin et al. 2008, Australian Journal of Crop Science 1(2): 43-46). For breeding methods in general see Principles of Plant Genetics and Breeding, 2007, George Acquaah, Blackwell Publishing, ISBN-13: 978-1-4051-3646-4.

The invention thus also provides a method of introducing a single locus conversion, or single trait conversion, into a white honeydew plant according to the invention and/or into NUN 01052 MEM comprising:

(a) crossing a white honeydew melon plant according to the invention, such as a melon plant of variety NUN 01052 MEM, a representative sample of seed of said variety having been deposited under Accession Number NCIMB ______, with a second melon plant comprising a desired single locus to produce F1 progeny plants;
(b) selecting F1 progeny plants that have the single locus to produce selected F1 progeny plants;
(c) crossing the selected progeny plants with at least a first white honeydew plant, e.g. with a plant of NUN 01052 MEM, to produce backcross progeny plants;
(d) selecting backcross progeny plants that have the single locus and one or more distinguishing characteristics of white honeydew melons according to the invention and/or the physiological and morphological characteristics of NUN 01052 MEM to produce selected backcross progeny plants; and
(e) repeating steps (c) and (d) one or more times in succession to produce selected second or higher backcross progeny plants that comprise the single locus and otherwise one or more distinguishing characteristics of white honeydew melons according to the invention and/or comprise all of the physiological and morphological characteristics of NUN 01052 MEM, when grown in the same environmental conditions.

The above method is provided, wherein the single locus confers a trait, wherein the trait is pest resistance or disease resistance.

In one embodiment the trait is disease resistance and the resistance is conferred to Bacterial Wilt, Root Rot, Crown Blight, Melon Rust, Powdery Mildew, Verticillum Wilt, Sulphur Burn, Scab, Watermelon Mosaic, Downy Mildew, Fusarium oxysporum f.sp. melonis (Fom) race 0, Fusarium oxysporum f.sp. melonis (Fom) race 1, Fusarium oxysporum f.sp. melonis (Fom) race 2, Fusarium oxysporum f.sp. melonis (Fom) race 1-2, Fusarium Wilt R2, Root Knot (Nematode), Anthracnose, Cucumber Mosiac, or Squash Mosaic.

In one embodiment the trait is pest resistance and the resistance is conferred to Aphid, Pickle Worm, Darkling Ground Beetle, Banded Cucumber Beetle, Mite, Western Spotted Cucumber Beetle, Melon Leafhopper, Melon Worm, Western Striped Cucumber Beetle or Melon Leafminer.

The invention also provides a melon plant comprising at least a first set of the chromosomes of melon line NUN 01052 MEM, a sample of seed of said line having been deposited under Accession Number NCIMB ______; further comprising a single locus conversion, wherein said plant has essentially all of the morphological and physiological characteristics of the plant comprising at least a first set of the chromosomes of melon line NUN 01052 MEM. In another embodiment, this single locus conversion confers a trait selected from the group consisting of male sterility, herbicide tolerance, insect resistance, pest resistance, disease resistance, environmental stress tolerance, modified carbohydrate metabolism and modified protein metabolism.

In one embodiment, NUN 01052 MEM may also be mutated (by e.g. irradiation, chemical mutagenesis, heat treatment, etc.) and mutated seeds or plants may be selected in order to change one or more characteristics of NUN 01052 MEM. Also natural mutants may be identified and used in breeding. Methods such as TILLING and/or EcoTILLING may be applied to melon populations in order to identify mutants. Similarly, NUN 01052 MEM may be transformed and regenerated, whereby one or more chimeric genes are introduced into the variety or into an EDV thereof. Transformation can be carried out using standard methods, such as Agrobacterium tumefaciens mediated transformation or biolistics, followed by selection of the transformed cells and regeneration into plants. A desired trait (e.g. genes conferring pest or disease resistance, herbicide, fungicide or insecticide tolerance, etc.) can be introduced into NUN 01052 MEM, or progeny thereof, by transforming NUN 01052 MEM or progeny thereof with a transgene that confers the desired trait, wherein the transformed plant retains all the phenotypic and/or morphological and/or physiological characteristics of NUN 01052 MEM or the progeny thereof and contains the desired trait.

The invention also provides for progeny of (white honeydew melons such as) melon variety NUN 01052 MEM obtained by further breeding with NUN 01052 MEM. In one aspect progeny are F1 progeny obtained by crossing NUN 01052 MEM with another plant or S1 progeny obtained by selfing NUN 01052 MEM. Also encompassed are F2 progeny obtained by selfing the F1 plants. “Further breeding” encompasses traditional breeding (e.g., selfing, crossing, backcrossing), marker assisted breeding, and/or mutation breeding. In one embodiment, the progeny have one or more of the distinguishing characteristics mentioned further above when grown under the same environmental conditions. In a further embodiment the progeny have all the physiological and morphological characteristics of variety NUN 01052 MEM when grown under the same environmental conditions. In another embodiment the progeny are EDVs and/or have one, two, or three distinct traits (qualitative or quantitative) introduced into NUN 01052 MEM, while retaining all the other physiological and morphological characteristics of variety NUN 01052 MEM when grown under the same environmental conditions.

White honeydew melons according to the invention, such as the variety NUN 01052 MEM, or its progeny (e.g. an EDV), can also be reproduced using vegetative reproduction methods. Therefore, the invention provides for a method of producing plants, or a part thereof, of a white honeydew plant, such as variety NUN 01052 MEM, comprising vegetative propagation of a white honeydew plant, such as variety NUN 01052 MEM. Vegetative propagation comprises regenerating a whole plant from a part of the white honeydew plant according to the invention, e.g. from variety NUN 01052 MEM, such as a cutting, a cell culture or a tissue culture.

The invention also provides for a vegetatively propagated plant of a white honeydew plant according to the invention, e.g. of variety NUN 01052 MEM, or a part thereof, having one or more distinguishing characteristics and/or all the morphological and physiological characteristics of NUN 01052 MEM, when grown under the same environmental conditions.

In one aspects haploid plants and/or double haploid plants of white honeydew plants according to the invention, such as NUN 01052 MEM, or an EDV or progeny of any of these, are encompassed herein. Haploid and double haploid (DH) plants can, for example, be produced by cell or tissue culture and chromosome doubling agents and regeneration into a whole plant. For DH production chromosome doubling may be induced using known methods, such as colchicine treatment or the like.

Also provided are plant parts derived from white honeydew plants according to the invention, such as from variety NUN 01052 MEM, or from a vegetatively propagated plant of NUN 01052 MEM, being selected from the group consisting of: harvested fruits or parts thereof, pollen, cells, leaves or parts thereof, petioles, cotyledons, hypocotyls, shoots or parts thereof, stems or parts thereof, roots or parts thereof, cuttings, or flowers.

In one embodiment, the invention provides for extracts of a plant described herein and compositions comprising or consisting of such extracts. In a preferred embodiment, the extract consists of or comprises tissue of a plant described herein or is obtained from such tissue.

The invention also provides for a food or feed product comprising or consisting of a plant part described herein preferably a melon fruit or part thereof and/or an extract from a plant part described herein. The food or feed product may be fresh or processed, e.g., canned, steamed, boiled, fried, blanched and/or frozen, etc.

For example, containers such as cans, boxes, crates, bags, cartons, Modified Atmosphere Packagings, films (e.g. biodegradable films), etc. comprising plant parts of plants (fresh and/or processed) described herein are also provided herein.

Marketable melon fruits are generally sorted by size and quality after harvest.

All documents (e.g., patent publications) are herein incorporated by reference in their entirety.

### EXAMPLES

### Development of NUN 01052 MEM

The hybrid NUN 01052 MEM was developed from a male and female proprietary inbred line of Nunhems. The male parent line was a double haploid line. The female and male parents were crossed to produce hybrid (F1) seeds of NUN 01052 MEM. The seeds of NUN 01052 MEM can be grown to produce hybrid plants and parts thereof (e.g. melon fruit). The hybrid NUN 01052 MEM can be propagated by seeds or vegetative.

The hybrid variety is uniform and genetically stable. This has been established through evaluation of horticultural characteristics. Several hybrid seed production events resulted in no observable deviation in genetic stability. Coupled with the confirmation of genetic stability of the female and male parents the Applicant concluded that NUN 01052 MEM is uniform and stable.

### Deposit Information

A total of 2500 seeds of the hybrid variety NUN 01052 MEM were deposited according to the Budapest Treaty by Nunhems B. V. on ______, at the American Type Culture Collection (ATCC), 10801 University Boulevard, Manassas, Va. 20110-2209 USA or at the NCIMB Ltd., Ferguson Building, Craibstone Estate, Bucksburn, Aberdeen AB21 9YA, United Kingdom (NCIMB). The deposit has been assigned Accession Number PTA ______or NCIMB ______.

A deposit of NUN 01052 MEM and of the male and female parent line is also maintained at Nunhems B. V. Access to the deposit will be available during the pendency of this application to persons determined by the Director of the U.S. Patent Office to be entitled thereto upon request. Subject to 37 C.F.R. §1.808(b), all restrictions imposed by the depositor on the availability to the public of the deposited material will be irrevocably removed upon the granting of the patent. The deposit will be maintained for a period of 30 years, or 5 years after the most recent request, or for the enforceable life of the patent whichever is longer, and will be replaced if it ever becomes nonviable during that period. Applicant does not waive any rights granted under this patent on this application or under the Plant Variety Protection Act (7 USC 2321 et seq.).

“USDA descriptors” are plant variety descriptors described for melon in the “Objective Description of Variety Muskmelon/Cantaloupe (Cucumis melo L.)—Exhibit C” of the U.S. Department of Agriculture, Agricultural Marketing Service, Science and Technology, Plant Variety Protection Office, Beltsville, Md. 20705, which can be downloaded from http://www.ams.usda.gov/AMSv1.0/ams.fetchTemplateData.do?template=TemplateJ&page=PV POForms, and is herein incorporated by reference in its entirety.

The most similar variety to NUN 01052 MEM are Sprite (comparison variety 1) for the flesh color, and Saturno (comparison variety 2) which is the most similar honeydew variety. In Table 1 a comparison between NUN 01052 MEM and Sprite/Saturno is shown based on a trial in the USA. Trial location: Acampo Calif. USA, (coordinates: 38°07′261″N, −121°18′80547″W), USA 2011. Average temperatures for the day were 33.1° C. and 14.7° C. for the night. Planting date: 20 Jun. 2011. Drip irrigation.

Two replication of 50 plants each, from which 20 plants or plant parts were randomly selected to measure characteristics. In Table 1 the USDA descriptors of the white honeydew melon according to the invention, NUN 01052 MEM (this application) and references Sprite and Saturno (both commercial varieties).

TABLE 1

| | Application Variety | Comparison | Comparison |
|---|---|---|---|
| | White honeydew | Variety 1 | Variety 2 |
| Descriptor | NUN 01052 MEM | Sprite | Saturno |
| 1. TYPE: | 2 | 6 | 2 |
| 1 = Persian 2 = Honey Dew | | | |
| 3 = Casaba 4 = Crenshaw | | | |
| 5 = Common or Summer | | | |
| 6 = Other Oriental. | | | |
| 2. AREA OF BEST | 3/4 | 1/4 | 3/4 |
| ADAPTATION IN U.S.A.: | | | |
| 1 = Southeast 2 = Northeast/ | | | |
| North Central 3 = Southwest | | | |
| 4 = Most Areas | | | |
| 3. MATURITY: | 65 | 70 | 76 |
| Days From Seeding to Harvest | | | |
| 4. PLANT: | | | |
| Fertility: | 1 | 1 | 1 |
| 1 = Andromonoecious 2 = | | | |
| Monoecious 3 = Gynoecious | | | |
| 4 = Other | | | |
| Habit: | 1 | 1 | 1 |
| 1 = Vine 2 = Semi-bush 3 = | | | |
| Bush | | | |
| 5. LEAF: | (Mature Blade of Third Leaf) | | |
| Shape: | 4 | 4 | 4 |
| 1 = Orbicular 2 = Ovate 3 = | | | |
| Reniform | | | |
| Lobes: | 2 | 2 | 2 |
| 1 = Not Lobed 2 = Shallowly | | | |
| Lobed 3 = Deeply Lobed | | | |
| Color: | 2 | 2 | 2 |
| 1 = Light Green (Honey Dew) | | | |
| 2 = Medium Green | | | |
| 3 = Dark Green (Rio Gold) | | | |
| Color Chart Name | RHS | RHS | RHS |
| Color Chart Code (GG = Green | GG 137 B | GG 137 A | YGG 147 B |
| Group; YGG = Yellow Green | | | |
| Group) | | | |
| Length mm | 14.0 | 14.6 | 13.4 |
| Width mm | 15.8 | 15.4 | 15.3 |
| Surface: | 3 | 3 | 3 |
| 1 = Pubescent 2 = Glabrous 3 = | | | |
| Scabrous | | | |
| 6. FRUIT: | (at Edible Maturity): | | |
| Length in cm | 13.7 | 10.3 | 13.5 |
| Diameter in cm | 11.3 | 9.3 | 12.7 |
| Weight gram | 1145 | 528 | 1474 |
| Shape: 1 = Oblate 2 = Oval | 2 | 2 | 2 |
| 3 = Round 4 = Elongate- | | | |
| Cylindrical 5 = Spindle 6 = | | | |
| Acorn | | | |
| Surface: 1 = Smooth 2 = | 1 | 1 - 2 | 1 - 2 |
| Netted 3 = Corrugated 4 = | | | |
| Warted | | | |
| Blossom Scar: | 1 | 2 | 2 |
| 1 = Obscure 2 = Conspicuous | | | |
| Rib Presence: | 1 | 1 | 1 |
| 1 = Absent 2 = Present | | | |
| No. Ribs per Fruit | N.A. | N.A. | N.A. |
| Rib Width at Medial in mm | N.A. | N.A. | N.A. |
| Ribs Surface: 1 = Smooth 2 = | N.A. | N.A. | N.A. |
| Netted | | | |
| Suture Depth: 1 = Shallow | N.A. | N.A. | N.A. |
| (Golden Delight) 2 = Medium | | | |
| 3 = Deep (Hackensack) | | | |
| Suture Surface: 1 = Smooth 2 = | N.A. | N.A. | N.A. |
| Netted | | | |
| Shipping Quality: 1 = Poor | 3 | 2 | 3 |
| (Home Garden) 2 = Fair (Short | | | |
| Distance Shipping) 3 = | | | |
| Excellent (Long Distance | | | |
| Shipping) | | | |
| Fruit Abscission: | 1 | 1 | 1 |
| 1 = When Ripe 2 = When | | | |
| Overripe 3 = Do Not Abscise | | | |
| 7. RIND NET: | | | |
| Net Presence: 1 = Absent 2 = | 1 | 2 | 2 |
| Sparse 3 = Abundant | | | |
| Distribution: 1 = Spotty 2 = | N.A. | 1 | 1 |
| Covers Entire Fruit | | | |
| Coarseness: 1 = Fine 2 = | N.A. | 1 | 1 |
| Medium Coarse 3 = Very | | | |
| Coarse | | | |
| Interlacing: 1 = None 2 = Some | N.A. | 1 | 1 |
| 3 = Complete | | | |
| Interstices: 1 = Shallow 2 = | N.A. | 1 | 1 |
| Medium Deep 3 = Deep | | | |
| 8. RIND TEXTURE: | | | |
| Texture: 1 = Soft 2 = Firm 3 = | 2 | 2 | 2 |
| Hard | | | |
| Thickness at Medial in mm | 3.6 | 2.6 | 3.0 |
| 9. RIND COLOR: | 01 = White 02 = Cream 03 = Buff 04 = Yellow 05 = | | |
| | Gold 06 = Green 07 = Orange 08 = Bronze 09 = | | |
| | Brown 10 = Gray 11 = Black 12 = Other | | |
| | (Specify) | | |
| Color Chart Name | RHS | RHS | RHS |
| Rind Color At Edible Maturity | | | |
| Primary Color/ | 02 | 02 | 02 |
| Color Chart Value | 11 D | 11 D | 11D |
| Mottling Color/ | N.A. | 04 | N.A. |
| Color Chart Value | N.A. | 5B | N.A. |
| Net Color/ | N.A. | 08 | 08 |
| Color Chart Value | N.A. | 163 B | 163 B |
| Furrow (Suture)/ | N.A. | N.A. | N.A. |
| Color Chart Value | N.A. | N.A. | N.A. |
| Rind Color At Full Maturity | | | |
| Primary Color/ | 02/04 | 04 | 04 |
| Color Chart Value | 2D | 12B | 2D |
| Mottling Color/ | N.A. | 04 | N.A. |
| Color Chart Value | N.A. | 5B | N.A. |
| Net Color/ | N.A. | 08 | 08 |
| Color Chart Value | N.A. | 164 B | 20 D |
| Furrow (Suture)/ | N.A. | N.A. | N.A. |
| Color Chart Value | N.A. | N.A. | N.A. |
| 10. FLESH (At Edible | 01 = White 02 = Cream 03 = Yellow 04 = Green 05 = | | |
| Maturity): | Orange 06 = Salmon 07 = Pink 08 = Other | | |
| | (Specify) | | |
| Color Chart Name | RHS | RHS | RHS |
| Color Near Cavity/ | 02 | 02 | 04 |
| Color Chart Value | 157 D | 155C | 142A |
| Color in Center/ | 02 | 02 | 04 |
| Color Chart Value | 157D | 155C | 142B |
| Color Near Rind/ | 02 | 02 | 04 |
| Color Chart Value | 157D | 155C | 142D |
| Refractometer % Soluable | 14.4 | 17.2 | 17.1 |
| Solids (Center of Flesh) | | | |
| Aroma: 1 = Absent 2 = Faint 3 = | 3 | 1 | 2 |
| Strong | | | |
| Flavor: 1 = Mild 2 = Somewhat | 2 | 1 | 1 |
| Spicy 3 = Very Spicy | | | |
| 11. SEED CAVITY: | | | |
| Length in mm | 94 | 68 | 85 |
| Width in mm | 50 | 45 | 50 |
| Shape in X-Section: | n.r. | n.r. | n.r. |
| 1 = Circular 2 = Triangular | | | |
| 12. SEEDS: | | | |
| No. Seeds per Fruit | 596 | 375 | 474 |
| gm per 1,000 Seeds | 26.3 | 13.8 | 40.0 |
| 13. DISEASE RESISTANCE: | (0 = Not Tested, 1 = Susceptible, 2 = Resistant) | | |
| Bacterial Wilt | 0 | 0 | 0 |
| Root Rot | 0 | 0 | 0 |
| Crown Blight | 0 | 0 | 0 |
| Melon Rust | 0 | 0 | 0 |
| Powdery Mildew | 2 | 0 | 0 |
| Verticillum Wilt | 0 | 0 | 0 |
| Sulphur Burn | 0 | 0 | 0 |
| Scab | 0 | 0 | 0 |
| Watermelon Mosaic | 0 | 0 | 0 |
| Downy Mildew | 0 | 0 | 0 |
| Fusarium Wilt R2 | 2 | 0 | 0 |
| Root Knot (Nematode) | 0 | 0 | 0 |
| Anthracnose | 0 | 0 | 0 |
| Cucumber Mosiac | 0 | 0 | 0 |
| Squash Mosaic | 0 | 0 | 0 |
| Other (specify) | 0 | 0 | 0 |
| 14. INSECT RESISTANCE: | (0 = Not Tested, 1 = Susceptible, 2 = Resistant) | | |
| Aphid | 0 | 0 | 0 |
| Pickle Worm | 0 | 0 | 0 |
| Darkling Ground Beetle | 0 | 0 | 0 |
| Banded Cucumber Beetle | 0 | 0 | 0 |
| Mite | 0 | 0 | 0 |
| Western Spotted Cucumber | 0 | 0 | 0 |
| Beetle | | | |
| Melon Leafhopper | 0 | 0 | 0 |
| Melon Worm | 0 | 0 | 0 |
| Western Striped Cucumber | 0 | 0 | 0 |
| Beetle | | | |
| Melon Leafminer | 0 | 0 | 0 |
| Other (Specify) | 0 | 0 | 0 |

These are typical values. Values may vary due to environment. Other values that are substantially equivalent are also within the scope of the invention. N.A.=not applicable; n.r.=not recorded.

### Example 2

### Texture and Chemical Analysis of Fruits

In 2010, the texture, organic acids and carbohydrates of NUN 01052 ME was compared to two green-fleshed honeydew melons, Santa Fe and Jade Delight. This was done at harvest (weeks 0), and after 2 weeks and three weeks of storage of the fruits at 10° C. (at about 95% relative humidity).

### Method

Five fruits of each variety were harvested at maturity and analysis was done on five fruits individually and the average value was calculated.

### Penetrometer Data:

Penetrometer data was obtained using a Fruit Pressure Tester model FT011 (QA Supplies) with an 8 mm diameter plunger (tip). The fruit was cut in half from the stem end to the blossom end. The measurement was taken on 3 locations on the cut surface, as indicated in the diagram below. The depth of plunger penetration to the fruit was 7 mm.

### Sugar Data:

Frozen melon was put into a plastic beaker and blended using a handheld blender until the sample becomes like powder. A portion of the ground sample was transferred to a 2-mL microcentrifuge tube, then centrifuged for 10 min at 15,000×g. The supernatant was diluted 1000-fold with MilliQ water (EMD Millipore) in a 50-mL conical centrifuge tube. One mL of the diluted extract was transferred into an HPLC vial. Sugars (glucose, fructose, and sucrose) were measured using ion-chromatography with pulsed amperometric detection (Dionex ICS-3000) on CarboPac PA1 analytical column (Dionex, 5.5 mm particle size, 3×250 mm), equipped with a guard column (Dionex, 5.5. mm particle size, 3×50 mm).

### Results

TABLE 2

| | | | | | ORGANIC ACIDS | | | | |
|---|---|---|---|---|---|---|---|---|---|
| | | CARBOHYDRATES | | | Citric | Isocitric | Malic | Succinic | TEXTURE |
| | | Glucose | Fructose | Sucrose | acid | acid | acid | acid | Penetro- |
| | Weeks | (mmol/ | (mmol/ | (mmol/ | (mmol/ | (mmol/ | (mmol/ | (mmol/ | meter |
| Variety | (10° C.) | g fwt) | g fwt) | g fwt) | g fwt) | g fwt) | g fwt) | g fwt) | (kg) |
| NUN01052 | 0 | 143.4 | 170.1 | 129.5 | 10.43 | 2.99 | 0.46 | 0.95 | 1.63 |
| | 2 | 166.0 | 224.4 | 184.5 | 17.86 | 4.97 | 4.45 | 1.18 | 1.60 |
| | 3 | 88.3 | 153.2 | 163.1 | 6.03 | 4.08 | 1.35 | 3.95 | 1.23 |
| Santa Fe | 0 | 96.3 | 153.4 | 195.9 | 15.19 | 5.60 | 0.00 | 2.07 | 1.63 |
| | 2 | 82.5 | 159.9 | 180.5 | 12.15 | 4.52 | 0.52 | 1.77 | 1.10 |
| | 3 | 83.1 | 181.3 | 191.3 | 10.73 | 4.99 | 0.40 | 2.12 | 1.15 |
| Jade | 0 | 187.1 | 225.9 | 243.8 | 13.90 | 3.67 | 0.05 | 1.73 | 2.71 |
| Delight | 2 | 118.8 | 179.9 | 198.8 | 15.14 | 5.43 | 0.18 | 2.65 | 1.49 |
| | 3 | 118.2 | 190.6 | 202.2 | 13.89 | 2.97 | 0.00 | 2.39 | 1.34 |

As can be seen, NUN01052 ME has excellent storage properties at 10 degrees Celsius. Fruit flesh texture (firmness) is essentially as firm after 2 weeks of storage at 10 degrees Celsius as it is at harvest, while in the other varieties decrease in flesh-firmness compared to harvest. Also the sugar and organic acid levels of NUN1052ME are very good two weeks after harvest, with fruits having higher average glucose, fructose and sucrose levels after two weeks of storage at 10 degrees Celsius compared to the levels at harvest. Likewise, citric acid, isocitric acid, malic acid and succinic acid levels were increased compared to harvest.

Thus, fruits have an average fruit firmness after at least about 14, 15, 16, 17, 18, 19, 20, 21, 22, 23, 24, or more, days storage at about 10° C. (preferably at a relative humidity, RH, of at least about 85%, preferably at least about 90% or 95% RH) which is essentially the same as the fruit firmness at harvest as determinable using a penetrometer (essentially the same meaning that the firmness measured after the period of storage is at least 95%, 96%, 97%, 98% or 99% of the firmness measured at harvest).

Fruits of NUN 01052ME remain essentially free of external blemishes and have a good eating quality until at least 14, 15, 16, 17, 18, 19, 20, 21, 22, 23, 24 or more days of storage at about 10° C. and a relative humidity of about 95%.

### Example 3

### Sensory Attributes of Fruits

Also the Sensory attributes at harvest were compared between NUN01052 ME and the two other green-fleshed honeydew melons, Santa Fe and Jade Delight. However, only data at harvest were collected. The results are shown in Table 3.

### Method

At maturity, 5 fruits of each variety were harvested from two replicates (10 fruits per variety in total). Seven trained test panelist tasted all 5 fruits from each variety. The average of the two replicates is provided, each replicate being an average of 7 panelists and 5 fruits (35 data points).

Sensory test was done by Scientific Certification Systems (SCS) in Emeryville, Calif., using 7 trained test panelists. Five melons per variety were used in this test. The sensory data consisted of 2 categories: attribute scores (fruity/melony, sweetness, juiciness, hardness, graininess) and acceptance scores (sweet/taste balance acceptance, texture acceptance, aroma acceptance, and overall quality acceptance). Each sensory data is an average of 7×5=35 data points.

The scoring was done on 0-15 scale.

For acceptance scores, 1-3=unacceptable, 4-6=below average, 7-9=average, 10-12=good, and 13-15=excellent.

For attribute scores, 0-2=trace, 3-6=light, 7-12=moderate, 13-15=strong.

### Results

TABLE 3

| | SENSORY at harvest | | | | | | | | |
|---|---|---|---|---|---|---|---|---|---|
| | | | | | | Sweet/Taste | | | Overall |
| | Fruity/ | Sweet- | Juici- | Hard- | Graini- | balance | Texture | Aroma | quality |
| | Melony | ness | ness | ness | ness | acceptance | acceptance | acceptance | acceptance |
| NUN01052 | 9.6 | 11.7 | 10.5 | 4.2 | 0.6 | 12.8 | 12.3 | 12.3 | 12.6 |
| Santa Fe | 10.4 | 10.4 | 10.5 | 3.6 | 1.0 | 11.4 | 10.9 | 11.9 | 11.4 |
| Jade | 9.8 | 13.3 | 10.6 | 6.8 | 0.2 | 13.2 | 12.0 | 11.8 | 12.2 |
| Delight | | | | | | | | | |

The results show that NUN01052 has good sensory attributes and especially and very good texture acceptance and overall eating quality acceptance at harvest. Especially, fruits according to the invention have an overall texture acceptance at harvest of at least 12.3 and/or an overall quality acceptance of at least 12.6 on a scale of 1-3=unacceptable, 4-6=below average, 7-9=average, 10-12=good, and 13-15=excellent.

1. A seed of a honeydew melon plant, which plant produced honeydew melon fruits having a cream colored fruit flesh and having an average fruit weight at edible maturity of at least 1000 grams.

2. The seed according to claim 1, wherein said cream color has a color chart value of RHS157D.

3. The seed according to claim 1 or 2, which plant produces fruits further comprising one or more characteristics selected from: the fruits have a non-netted rind and fruits having a rind thickness at medial of at least about 3 mm, preferably 3.2 mm, 3.3 mm, 3.4 mm or more.

4. The seed according to any one of claims 1 to 3, which plant produces fruits further comprising one or more characteristics selected from: a) fruits have a strong fruit aroma wherein fruit aroma is selected from the categories: absent, faint and strong, b) fruits have a ‘tropical’ flavor, resembling a pineapple/coconut/banana flavor, c) fruits having an average fruit firmness after 14 days of storage at about 10° C. which is at least 95% of the firmness measured at harvest and d) the number of days from seeding to harvest are at least 10 days less than for variety Saturno.

5. The seed according to any one of claims 1 to 3, which plant produces fruits having an average fruit firmness after 14 days of storage at about 10° C. which is at least 95% of the firmness measured at harvest.

6. A seed of melon variety NUN 01052 MEM, wherein a representative sample of said seed has been deposited under Accession Number NCIMB ______.

7. A plant of melon variety NUN 01052 MEM, or a part thereof, wherein a representative sample of seed of said variety has been deposited under Accession Number NCIMB ______.

8. A fruit of melon variety NUN 01052 MEM, or a plant part produced from the plant of claim 7.

9. A method of producing a melon plant, comprising crossing the plant of claim 1 or 7 with a second melon plant one or more times, and selecting progeny from said crossing.

10. A method of producing a melon plant, comprising selfing the plant of claim 1 or 7 one or more times, and selecting progeny from said selfing.

11. Progeny of a melon plant according to claim 1 or progeny of melon variety NUN 01052 MEM obtained by further breeding with said variety, wherein said progeny produces honeydew melon fruits with cream colored fruit flesh and an average fruit weight at edible maturity of at least 1000 grams.

12. The progeny of claim 11, wherein said progeny have all the physiological and morphological characteristics of melon variety NUN 01052 MEM when grown under the same environmental conditions.

13. An Essentially Derived Variety of NUN 01052 MEM having one, two or three physiological and/or morphological characteristics which are different from those of NUN 01052 MEM and which otherwise has all the physiological and morphological characteristics of NUN 01052 MEM, wherein a representative sample of seed of variety NUN 01052 MEM has been deposited under Accession Number NCIMB ______.

14. A method of producing plants, or a part thereof, of variety NUN 01052 MEM comprising vegetative propagation of variety NUN 01052 MEM.

15. The method of claim 14, wherein said vegetative propagation comprises regenerating a whole plant from a part of variety NUN 01052 MEM.

16. A vegetative propagated plant of variety NUN 01052 MEM, or a part thereof, having all the morphological and physiological characteristics of NUN 01052 MEM when grown under the same environmental conditions.

17. Plant part derived from variety NUN 01052 MEM, or from a plant of claim 12, wherein said plant part are harvested fruit or parts thereof, pollen, cells, leaves or parts thereof, petioles, shoots or parts thereof, stems or parts thereof, roots or parts thereof, cuttings, or flowers or parts thereof.

18. A food or feed product comprising a plant part of claim 17.

19. The food or feed product of claim 18, wherein said plant part is fresh or processed.

20. A melon plant or melon fruit produced by growing the seed of claim 1 or claim 5.

21. A cell or tissue culture produced from a plant of claim 1 or 5.

22. A melon plant regenerated from a cell or tissue culture of claim 21.

23. A melon plant comprising at least a first set of the chromosomes of melon line NUN 01052 MEM, a sample of seed of said line having been deposited under Accession Number NCIMB ______ and further comprising a single locus conversion, wherein said plant has essentially all of the morphological and physiological characteristics of the plant comprising at least a first set of the chromosomes of melon line NUN 01052 MEM.

24. The plant of claim 23, wherein the single locus conversion confers a trait selected from the group consisting of male sterility, herbicide tolerance, insect resistance, pest resistance, disease resistance, environmental stress tolerance, modified carbohydrate metabolism and modified protein metabolism.
